(19) United States
(12) Patent Application Publication
(10) Pub. No.: US 2024/0075291 A1
(43) Pub. Date: Mar. 7, 2024

(54) CLOSE-LOOP DEEP BRAIN STIMULATION ALGORITHM SYSTEM FOR PARKINSON?S DISEASE AND CLOSE-LOOP DEEP BRAIN STIMULATION ALGORITHM METHOD FOR PARKINSON?S DISEASE

(71) Applicant: Industrial Technology Research Institute, Hsinchu (TW)

(72) Inventors: Chii-Wann Lin, Taipei City (TW); Chia-Hung Cho, Taichung City (TW)

(73) Assignee: Industrial Technology Research Institute, Hsinchu (TW)

(21) Appl. No.: 18/088,777

(22) Filed: Dec. 27, 2022

Related U.S. Application Data
(60) Provisional application No. 63/403,294, filed on Sep. 1, 2022.

(51) Int. Cl. A61N 1/36

(52) U.S. Cl. CPC A61N 1/36067; A61N 1/36139; A61N 1/36178

(57) ABSTRACT

A close-loop deep brain stimulation algorithm system for Parkinson's disease includes a memory and a processor. The processor includes a deep brain stimulation (DBS) simulation module, a virtual brain network module, a feature extraction module, and a reinforcement learning module. The deep brain stimulation simulation module is adapted to combine a deep brain stimulation waveform according to the stimulation frequency and the stimulation amplitude and output the deep brain stimulation waveform. The virtual brain network module is adapted to receive the deep brain stimulation waveform to output a synaptic signal and calculate a reward parameter. The feature extraction module is adapted to receive the synaptic signal and extract a plurality of feature values according to the synaptic signal. The reinforcement learning module is adapted to train a deep brain stimulation neural network based on the feature values and reward parameter and output the stimulation frequency and the stimulation amplitude to the deep brain stimulation simulation module.

### CROSS-REFERENCE TO RELATED APPLICATION

This application claims the priority benefit of U.S. provisional application Ser. No. 63/403,294, filed on Sep. 1, 2022. The entirety of the above-mentioned patent application is hereby incorporated by reference herein and made a part of this specification.

### TECHNICAL FIELD

The disclosure relates to a deep brain stimulation algorithm technique, and in particular relates to a close-loop deep brain stimulation algorithm system for Parkinson's disease and a close-loop deep brain stimulation algorithm method for Parkinson's disease.

### BACKGROUND

Parkinson's disease (PD) is a chronic neurodegenerative disease that affects the central nervous system, less than Alzheimer's disease, the currently affected population is about 10 million people worldwide. The application of deep brain stimulation (DBS) technology in movement disorders and neurological diseases, including Parkinson's disease, tremor, dystonia, epilepsy, obsessive-compulsive disorder, etc., has been proved to be an effective treatment.

However, the widely used open-loop stimulation system still has some shortcomings that need to be corrected. For example, the open-loop stimulation system is highly dependent on the individual, and has the characteristics of high energy consumption, frequent clinic visiting, and trial-and-error parameter adjustments. The strategy of the currently known close-loop stimulation system adopts discriminative signals or biomarkers, so that the system may automatically adjust the parameters of deep brain stimulation through algorithms.

However, the sensors in the conventional close-loop stimulation system must detect the thalamic action potential at any time. Once the thalamic action potential has an abnormal erroneous response, it is necessary to electrically stimulate the subthalamic nucleus region of the brain with deep brain stimulation current, causing the battery life of the conventional close-loop stimulation system to be less than that of the open-loop stimulation system. Therefore, how to achieve a better thalamus relay repair effect and save energy consumption will be a subject that requires breakthroughs.

### SUMMARY

This disclosure provides a close-loop deep brain stimulation (DBS) algorithm system for Parkinson's disease, including a memory and a processor. The memory stores the deep brain stimulation neural network; the processor is coupled to the memory. The processor includes a deep brain stimulation simulation module, a virtual brain network module, a feature extraction module, and a reinforcement learning module. The deep brain stimulation simulation module is adapted to combine a deep brain stimulation waveform according to a stimulation frequency and a stimulation amplitude, and output the deep brain stimulation waveform. The virtual brain network module is adapted to receive the deep brain stimulation waveform to output a synaptic signal and calculate a reward parameter. The feature extraction module is adapted to receive the synaptic signal and extract multiple feature values according to the synaptic signal. The reinforcement learning module is adapted to train the deep brain stimulation neural network based on the feature values and the reward parameter, and output the stimulation frequency and stimulation amplitude to the deep brain stimulation simulation module.

This disclosure provides a close-loop deep brain stimulation algorithm method for Parkinson's disease, including the following operation. A deep brain stimulation waveform is combined through a deep brain stimulation simulation module according to a stimulation frequency and a stimulation amplitude, and the deep brain stimulation waveform is output. The deep brain stimulation waveform is received through a virtual brain network module to output a synaptic signal and calculate a reward parameter. The synaptic signal is received through a feature extraction module, and multiple feature values are extracted according to the synaptic signal. A deep brain stimulation neural network is trained through a reinforcement learning module based on the feature values and the reward parameter, and the stimulation frequency and stimulation amplitude are output to the deep brain stimulation simulation module.

### BRIEF DESCRIPTION OF THE DRAWINGS

FIG. 1 is an architectural diagram of a close-loop deep brain stimulation algorithm system for Parkinson's disease according to an embodiment of the disclosure.

FIG. 2A is a signal schematic diagram of a thalamic action potential in a normal state in a close-loop deep brain stimulation algorithm system for Parkinson's disease according to an embodiment of the disclosure.

FIG. 2B is a signal schematic diagram of a Parkinson's state without applying deep brain stimulation in a close-loop deep brain stimulation algorithm system for Parkinson's disease according to an embodiment of the disclosure.

FIG. 2C is a signal schematic diagram of a Parkinson's state applying deep brain stimulation in a close-loop deep brain stimulation algorithm system for Parkinson's disease according to an embodiment of the disclosure.

FIG. 3 is an architectural diagram of a close-loop deep brain stimulation algorithm system for Parkinson's disease connected to a subject brain according to an embodiment of the disclosure.

FIG. 4 is a flowchart of a close-loop deep brain stimulation algorithm method for Parkinson's disease according to an embodiment of the disclosure.

### DETAILED DESCRIPTION OF DISCLOSED EMBODIMENTS

A portion of the embodiments of the disclosure will be described in detail with reference to the accompanying drawings. Element symbol referenced in the following description will be regarded as the same or similar element when the same element symbol appears in different drawings. These examples are only a portion of the disclosure and do not disclose all possible embodiments of the disclosure.

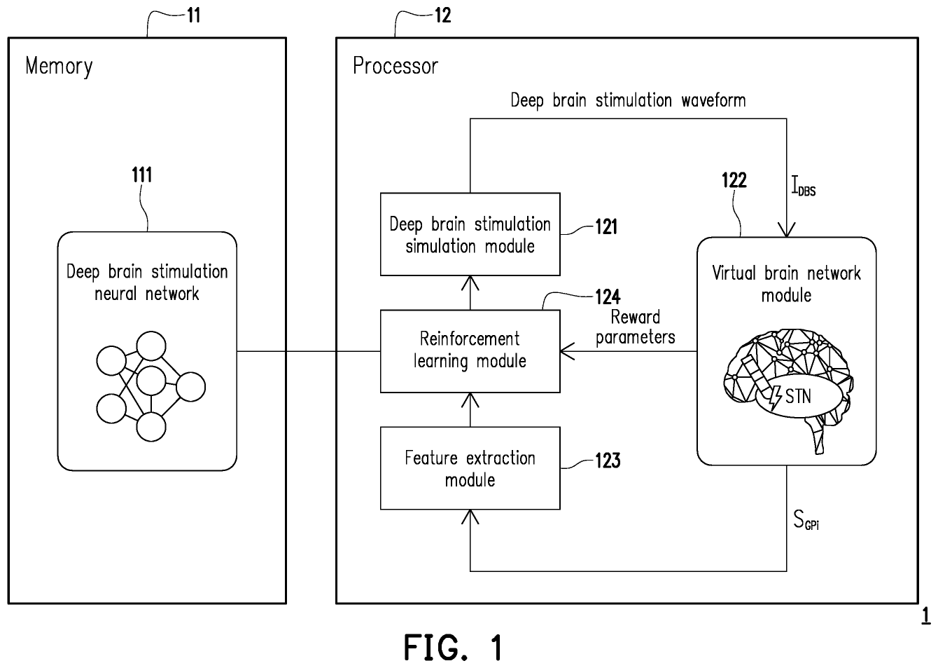

FIG. 1 is an architectural diagram of a close-loop deep brain stimulation algorithm system 1 for Parkinson's disease according to an embodiment of the disclosure. Referring to FIG. 1, the close-loop deep brain stimulation algorithm system 1 for Parkinson's disease includes a memory 11 and a processor 12. The memory 11 is adapted to store a deep brain stimulation neural network 111, and the processor 12 is coupled to the memory 11.

Practically speaking, the close-loop deep brain stimulation algorithm system 1 for Parkinson's disease may be implemented by computer devices, such as desktop computers, notebook computers, tablet computers, workstations, etc. with computing functions, display functions and networking functions, the disclosure is not limited thereto. The memory 11 is, for example, a static random-access memory (SRAM), a dynamic random-access memory (DRAM) or other memories. The processor 12 may be a central processing unit (CPU), a micro-processor, or an embedded controller, which is not limited in this disclosure.

The processor 12 includes a deep brain stimulation simulation module 121, a virtual brain network module 122, a feature extraction module 123, and a reinforcement learning module 124.

The deep brain stimulation simulation module 121 combines a deep brain stimulation waveform according to a stimulation frequency and a stimulation amplitude, and outputs the deep brain stimulation waveform. The deep brain stimulation waveform output by the deep brain stimulation simulation module 121 is a biphasic, symmetrical, and charge-balanced pulse wave, and the pulse width may be, for example, 60 μs.

When the existing open-loop or close-loop brain stimulator is connected to the actual brain, the subthalamic nucleus region in the actual brain is electrically stimulated with the deep brain stimulation current I_{DBS}. Therefore, the deep brain stimulation waveform in this disclosure is a deep brain stimulation current I_{DBS} adapted to simulate the stimulation of the subthalamic nucleus region in the actual brain by a brain stimulator.

The virtual brain network module 122 receives the deep brain stimulation waveform to output the synaptic signal S_{GPi}, calculates the reward parameter, and stores the reward parameter to the memory 11.

In practice, this disclosure may establish a basal ganglia-thalamic (BGT) brain network through Python or other programming languages to implement the virtual brain network module 122 and construct a network environment that simulates the brain. In detail, this disclosure mainly simulates four types of nerve cells in the basal ganglia-thalamic (BGT) nerve, namely the subthalamic nucleus (STN) neuron, the inner globus pallidus (GPi) neuron, the external globus pallidus (GPe) neuron, and the thalamic (TH) neuron, and each nucleus contains 10 neurons. Each neuron simulates neuronal behavior based on a Hodgkin-Huxley model that describes how action potentials are generated and conducted in neurons. The neurons in each part also reaches three different state systems through the dynamic balance of inhibitory and excitatory synaptic connections/coupling: a normal state, a Parkinson's state, and a 130 Hz or other frequency electrical stimulation to the subthalamic nucleus neurons in the Parkinson's state.

In addition, this disclosure uses an OpenAI Gym architecture to design the interactive interface between the virtual brain network module 122 and the reinforcement learning module 124, so that related signals such as the action space, state space, reward parameter, the start and end conditions of the signal surge event, the step size, and the time window are sent to the reinforcement learning module 124.

The state space output by the virtual brain network module 122 includes multiple feature values extracted from the synaptic signal S_{GPi}, and the feature values are extracted by the feature extraction module 123 and serve as input values sent by the feature extraction module 123 to the reinforcement learning module 124. In addition, the reward parameter output by the virtual brain network module 122 assists the reinforcement learning module 124 to train the deep brain stimulation neural network 111 in order to find the suitable deep brain stimulation waveform, that is, the stimulation frequency and stimulation amplitude, for any input state.

This disclosure randomly selects a state (environmental index) from the normal state and the Parkinson's state at the beginning of a signal surge event, so as to assist the deep brain stimulation neural network 111 to generalize the scenarios under normal conditions and Parkinson's disease pathological conditions, and this random effect does not affect the model training of the deep brain stimulation neural network 111. The step size of the output of the virtual brain network module 122 is set to 100 ms, that is, the time window of an action and state is 100 ms without overlapping. The action space output by the virtual brain network module 122 is formed by a two-dimensional stimulation frequency and stimulation amplitude, which serve as the output of the reinforcement learning module 124 and the input of the virtual brain network module 122. The stimulation frequency range may be, for example, 100 to 185 Hz, and the stimulation amplitude range may be, for example, 0 to 5000 μA/cm 2.

In addition, the synaptic signal S_{GPi} belongs to the deep brain nerve cell signal, which is difficult to be directly recorded in the actual brain environment. It is essentially hidden in the superficial extracellular signal of the actual brain environment (e.g., EEG signal, local field potential, etc.). Therefore, in this disclosure, the synaptic signal S_{GPi} first extracts multiple feature values through the feature extraction module 123, which serve as the input of the reinforcement learning module 124. In this disclosure, the internal globus pallidus synaptic signal is selected as the biomarker signal for training, and the feature extraction module 123 designed based on the extracellular electrophysiological signal serves as a mapping tool between the virtual thalamic action potential signal of the virtual brain network module 122 and the extracellular signal from the actual brain, thereby allowing future testing in animal experiments and clinical trials.

The feature extraction module 123 receives the synaptic signal S_{GPi}, extracts multiple feature values according to the synaptic signal Sm, and stores the feature values into the memory 11. The total dimension of the feature values extracted by the feature extraction module 123 in this disclosure is 5, and the feature values extracted by the feature extraction module 123 are further described in detail below.

The feature values described in this disclosure include the Hjorth parameter. The Hjorth parameter is a statistical characteristic that characterizes the EEG time-domain signal, including three types of parameter indicators: the Hjorth activity indicator, the Hjorth mobility indicator, and the Hjorth complexity indicator, respectively representing the average power, the average frequency, and the frequency change of the signal. Assuming y(t) is a time-domain signal, the formulas of the Hjorth activity, the Hjorth mobility, and the Hjorth complexity are:

$$Activity ( y ( t ) ) = var ( y ( t ) ) \qquad ( 1 )$$

$$Mobility ( y ( t ) ) = \sqrt{var ( \frac{d y ( t )}{d t} ) / var ( y ( t ) )} \qquad ( 2 )$$

$$Complexity ( y ( t ) ) = \frac{Mobility ( \frac{d y ( t )}{d t} )}{Mobility ( y ( t ) )} \qquad ( 3 )$$

The feature values described in this disclosure further include β band power and sample entropy. In the local field potential (LFP) recorded from subthalamic nucleus neurons in patients with Parkinson's disease, β band (12 to 30 Hz) oscillatory power is associated with movement disorders (bradykinesia and rigidity). High levels of power are present in both subthalamic nucleus neurons and internal global pallidus neurons in patients with Parkinson's disease, which may be inhibited by sufficient stimulation or medication. This disclosure uses the SciPy package to estimate the power spectral density, calculates the definite integral of the area in the expected frequency band according to the trapezoidal rule, and sums up the power obtained from the synaptic signal S_{GPi} of each inner global pallidus neuron to serve as the β band power.

In addition, the sample entropy has been applied to assess the complexity of physiological time series signals and diagnose the disease state with low computational complexity and independence to the data degree. Smaller entropy represents a higher degree of self-similarity or lower complexity and irregularity in the data. In the case of a patient with Parkinson's disease, synchronous oscillations begin to appear among the neural nuclei, and the sample entropy value is significantly different from the normal state. Therefore, this disclosure also lists the sample entropy as one of the extracted feature values.

In the disclosure, the end condition of a signal surge event may be regarded as the achievement of a short-term goal, for example, when the β band power serving as one of the feature values is suppressed below a threshold value, and the thalamic error index (EI) is 0, then the signal surge event is set to end. Generally speaking, the threshold value is approximately set at the β band power in a normal state, and may be fine-tuned depending on the training situation, but not limited thereto.

The reinforcement learning module 124 trains the deep brain stimulation neural network 111 stored in the memory 11 based on the feature values and the reward parameter, and outputs the stimulation frequency and the stimulation amplitude to the deep brain stimulation simulation module 121. The deep brain stimulation simulation module 121 combines a deep brain stimulation waveform according to the stimulation frequency and stimulation amplitude output by the reinforcement learning module 124, and outputs the deep brain stimulation waveform to the virtual brain network module 122. After receiving the deep brain stimulation waveform, the virtual brain network module 122 calculates the reward parameter and outputs the synaptic signal S_{GPi} to the feature extraction module 123. The feature extraction module 123 extracts multiple feature values according to the synaptic signal S_{GPi}, and the reinforcement learning module 124 then trains the deep brain stimulation neural network 111 based on the feature values and the reward parameter.

The deep brain stimulation neural network 111 is continuously trained through the reinforcement learning module 124, so that the reinforcement learning module 124 may quickly find the suitable stimulation parameters (the stimulation frequency and stimulation amplitude) in any input state, thereby the deep brain stimulation simulation module 121 may output the suitable deep brain stimulation waveform under suitable conditions. In one embodiment, the reinforcement learning module 124 is a twin-delayed deep deterministic policy gradient (TD3) architecture.

The reward parameter described in this disclosure may be calculated by the virtual brain network module 122 according to the thalamic error index (EI), and the thalamic error index EI is related to the brain cortical signal and the thalamic action potential signal. Specifically, the thalamic error index EI is the ratio of the number of erroneous pulses of the thalamic action potential signal to the number of pulses of the brain cortical signal. In the close-loop deep brain stimulation algorithm system 1 for Parkinson's disease described in this disclosure, the virtual brain network module 122 is more adapted to generate virtual brain cortical signals and virtual thalamic action potential signals.

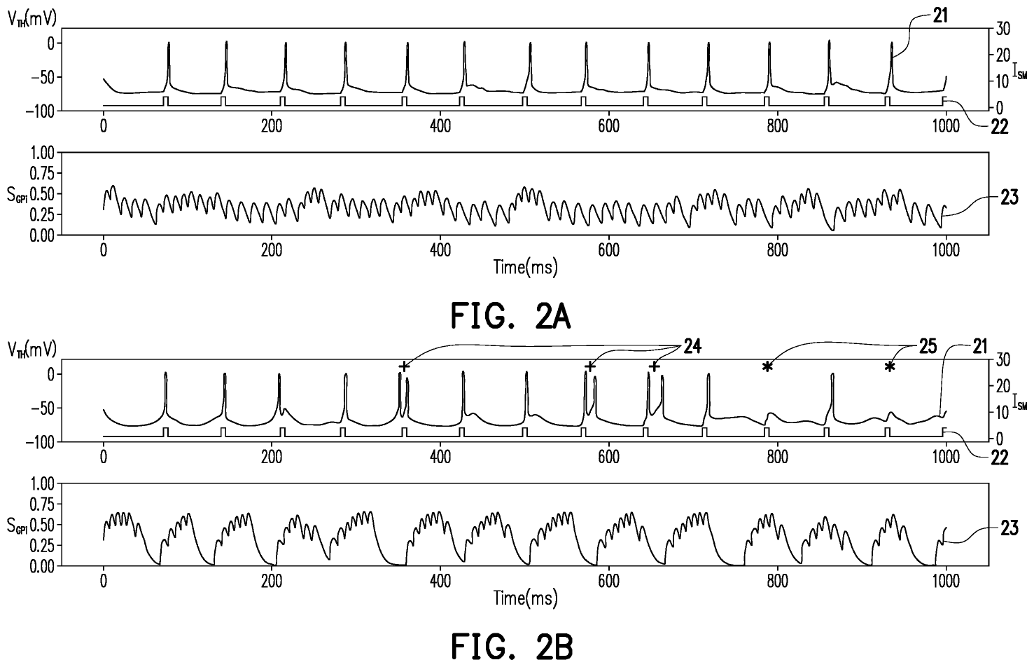

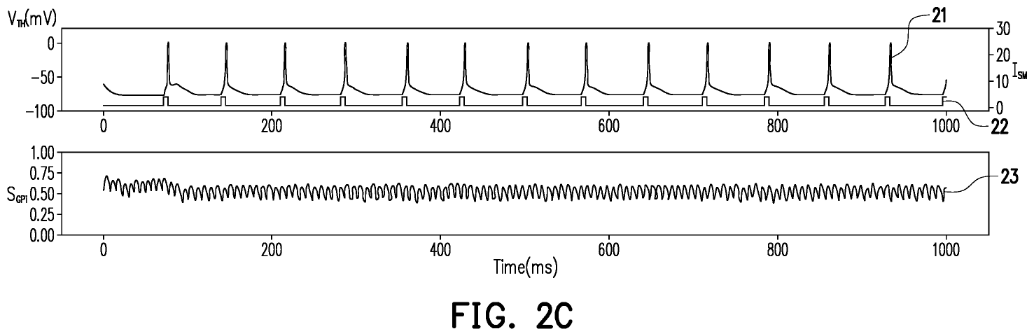

FIG. 2A is a signal schematic diagram of a thalamic action potential in a normal state in a close-loop deep brain stimulation algorithm system for Parkinson's disease according to an embodiment of the disclosure. As shown in FIG. 2A, under the normal state of thalamic action potential, the virtual thalamic action potential signal 21 stably generates a single action potential along with the pulse of the virtual brain cortical signal 22, and the synaptic signal 23 is also stably output from the brain due to virtual thalamic action potential signal 21.

The thalamic action potential of patients with Parkinson's disease have a surge signal erroneous response. The close-loop deep brain stimulation algorithm system 1 for Parkinson's disease described in this disclosure may calculate the thalamic error index through the virtual brain cortical signal and the virtual thalamic action potential signal generated by the virtual brain network module 122. FIG. 2B is a signal schematic diagram of a Parkinson's state without applying deep brain stimulation in a close-loop deep brain stimulation algorithm system for Parkinson's disease according to an embodiment of the disclosure. As shown in FIG. 2B, when the deep brain stimulation simulation module 121 does not apply deep brain stimulation waveforms to the virtual brain network module 122, the virtual thalamic action potential signal 21 generated by the virtual brain network module 122 may have a surge erroneous response. The mark “+” 24 represents the surge erroneous response, that is, the pulse of the virtual thalamic action potential signal 21 has more than one action potential, and the mark “*” 25 represents the erroneous response of a missing pulse, that is, the pulse of the virtual thalamic action potential signal 21 does not form a single action potential. In the abnormal state of the thalamic action potential, the virtual thalamic action potential signal 21 generates more than one action potential or fails to form an action potential due to the unstable pulse of the virtual brain cortical signal 22, and the synaptic signal 23 also cannot be output stably from the brain.

As mentioned above, the thalamic error index EI is the ratio of the number of erroneous pulses of the thalamic action potential signal to the number of pulses of the brain cortical signal. As shown in FIG. 2B, assuming that the number of pulses of the brain cortical signal is 10, and the number of erroneous pulses of the thalamic action potential signal (including the mark “+”24 and the mark “*” 25) is 4, the thalamic error index EI is 0.4.

FIG. 2C is a signal schematic diagram of a Parkinson's state applying deep brain stimulation in a close-loop deep brain stimulation algorithm system for Parkinson's disease according to an embodiment of the disclosure. As shown in FIG. 2C, since the deep brain stimulation simulation module 121 applies a deep brain stimulation waveform to the virtual brain network module 122 when the virtual thalamic action potential signal 21 has a surge erroneous response, the virtual brain network module 122 receives the deep brain stimulation waveform to output the synaptic signal S_{GPi}, and the virtual thalamic action potential signal 21 may stably generate a single action potential. Therefore, in the Parkinson's state simulated by the virtual brain network module 122, the deep brain stimulation waveform is applied to the virtual brain network module 122 through the deep brain stimulation simulation module 121, the virtual thalamic action potential signal 21 does not have the mark “+” 24 or the mark “*” 25, and the thalamic error index EI is 0, which is the same as in the normal state.

Once the virtual brain network module 122 calculates the thalamic error index EI, the reward parameter described in this disclosure may be calculated according to the thalamic error index EI through the virtual brain network module 122. The reward parameter described in this disclosure are further explained below.

The reward parameter R(t) described in this disclosure is related to the revised score, deep brain stimulation energy expenditure penalty, current state penalty, and compensation score. In the design of the reward parameter R(t), it is mainly formed by the following four formulas: the revised score r₁, the deep brain stimulation energy expenditure penalty r₂, the current state penalty r₃, and the compensation score r₄.

The revision score r₁, (EIₜ₋₁−EIₜ), where EIₜ₋₁ and EIₜ are thalamic error indices EI before and after the deep brain stimulation simulation module 121 applies the deep brain stimulation current I_{DBS} (or the deep brain stimulation waveform) respectively.

The deep brain stimulation energy expenditure penalty

$${r}_{2} = - \sqrt{\frac{1}{T} {\int }_{0}^{T} {I}_{D B S}^{2} ( t ) d t}$$

The current state penalty

$${r}_{3} = \{ \begin{matrix}- {EI}_{t} , & if {r}_{1} \leq 0 \\ 0 , & otherwise\end{matrix} ,$$

where the current state penalty r₃ may guide the model of the deep brain stimulation neural network 111 to meet the end condition of the signal surge event as soon as possible.

The compensation score

$${r}_{4} = \{ \begin{matrix}1 , & if {r}_{1} ∩ {r}_{2} ∩ {r}_{3} = 0 \\ 0 , & otherwise\end{matrix} ,$$

where the purpose of the compensation score r₄ is to compensate the score for the reinforcement learning module 124 when the deep brain stimulation is turned off in a normal state, so as to encourage the model of the deep brain stimulation neural network 111 to save energy.

Through the aforementioned four formulas, the reward parameter R(t) is obtained by a sum of each of the revised score r₁, the deep brain stimulation energy expenditure penalty r₂, the current state penalty r₃, and the compensation score r₄ multiplied by their respective weight parameters, namely the reward parameter R(t), λ₁r₁+λ₂r₂+λ₃r₃+λ₄r₄, and in this embodiment the weight parameter λ₁=15, λ₂=5×10⁻⁴, λ₃=3, λ₄=2. It should be noted here that these weight parameters may be adjusted according to actual conditions, and this disclosure is not limited thereto.

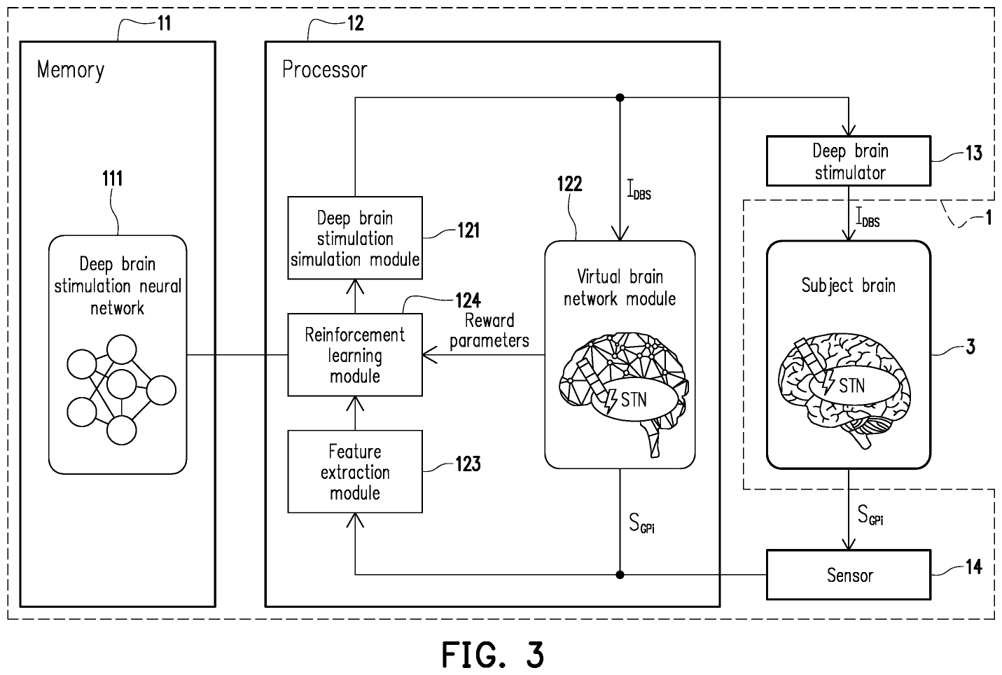

As mentioned above, the feature extraction module 123 described in this disclosure may serves as a mapping tool between the virtual thalamic action potential signal of the virtual brain network module 122 and the extracellular signal from the actual brain, thereby allowing future testing in animal experiments and clinical trials. FIG. 3 is an architectural diagram of a close-loop deep brain stimulation algorithm system 1 for Parkinson's disease connected to a subject brain 3 according to an embodiment of the disclosure. As shown in FIG. 3, the close-loop deep brain stimulation algorithm system 1 for Parkinson's disease further includes a deep brain stimulator 13 and a sensor 14. The deep brain stimulator 13 is connected to the deep brain stimulation simulation module 121 in the processor 12 and the subject brain 3, and the sensor 14 is connected to the feature extraction module 123 in the processor 12 and the subject brain 3. In one embodiment, the sensor 14 is more adapted to sense the brain cortical signal and the thalamic action potential signal of the subject brain 3.

The deep brain stimulator 13 uses the stimulation frequency and stimulation amplitude output by the deep brain stimulation simulation module 121 in the processor 12 as the deep brain stimulation waveform to generate the deep brain stimulation current I_{DBS} corresponding to the deep brain stimulation waveform, and stimulates the subject brain 3 with the deep brain stimulation current I_{DBS}. The sensor 14 senses the synaptic signal S_{GPi} output by the subject brain 3.

The feature extraction module 123 receives the synaptic signal Sm output from the sensor 14, and extracts the feature values according to the synaptic signal Sm. The reinforcement learning module 124 outputs the stimulation frequency and stimulation amplitude to the deep brain stimulation module 121 through the trained deep brain stimulation neural network 111. The deep brain stimulation module 121 outputs the deep brain stimulation waveform to the deep brain stimulator 13, and uses the deep brain stimulation current I_{DBS} to electrically stimulate the subthalamic nucleus region in the subject brain 3.

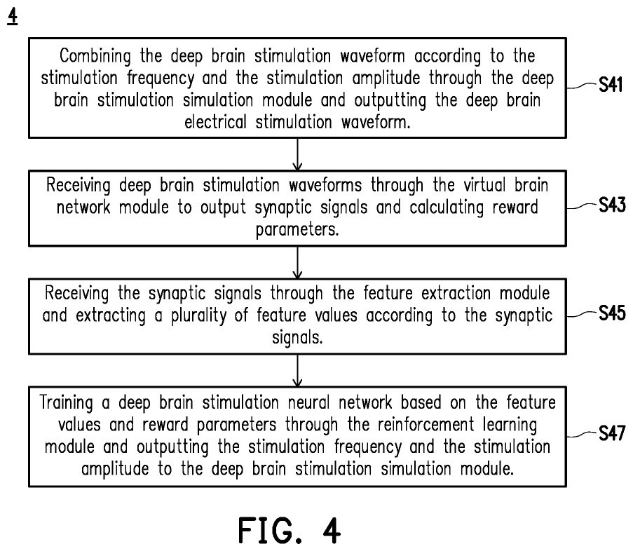

FIG. 4 is a flowchart of a close-loop deep brain stimulation algorithm method 4 for Parkinson's disease according to an embodiment of the disclosure. The close-loop deep brain stimulation algorithm method 4 for Parkinson's disease includes step S41, step S43, step S45, and step S47.

In step S41, the deep brain stimulation waveform is combined through the deep brain stimulation simulation module according to the stimulation frequency and the stimulation amplitude, and the deep brain stimulation waveform is output. In one embodiment, the deep brain stimulation waveform is a biphasic pulse wave. In step S43, the deep brain stimulation waveform is received through the virtual brain network module to output a synaptic signal, and a reward parameter is calculated. In step S45, the synaptic signal is received through the feature extraction module, and multiple feature values are extracted according to the synaptic signal. In step S47, the deep brain stimulation neural network is trained through the reinforcement learning module based on the feature values and the reward parameter, and the stimulation frequency and the stimulation amplitude are output to the deep brain stimulation simulation module.

In one embodiment, the close-loop deep brain stimulation algorithm method for Parkinson's disease further includes generating the virtual brain cortical signal and the virtual thalamic action potential signal through the virtual brain network module, and calculating the thalamic error index according to the virtual cortical signal and the virtual thalamic action potential signal.

In one embodiment, the close-loop deep brain stimulation algorithm method for Parkinson's disease further includes calculating the reward parameter through the virtual brain network module according to the thalamic error index. The reward parameter is related to revised score, the deep brain stimulation energy expenditure penalty, current state penalty, and compensation score. The reward parameter is obtained by a sum of each of the revised score, the deep brain stimulation energy expenditure penalty, the current state penalty, and the compensation score multiplied by their respective weight parameters. The details about the thalamic error index, reward parameter, revised score, the deep brain stimulation energy expenditure penalty, current state penalty, and compensation score have been described in detail in the previous paragraphs, and are not repeated herein.

In one embodiment, the feature values described in the close-loop deep brain stimulation algorithm for Parkinson's disease include Hjorth parameters, β band power and sample entropy, in which the Hjorth parameter includes a Hjorth activity indicator, a Hjorth mobility indicator, and a Hjorth complexity indicator. The reinforcement learning module is a twin-delayed deep deterministic policy gradient architecture. The details about the Hjorth activity indicator, the Hjorth mobility indicator, the Hjorth complexity indicator, the β band power, and the sample entropy have been described in detail in the previous paragraphs, and are not repeated herein.

In one embodiment, when the deep brain stimulation simulation module and the feature extraction module are connected to the subject brain, the close-loop deep brain stimulation algorithm method for Parkinson's disease further includes the following operation. The deep brain stimulation current is generated through the deep brain stimulator according to the stimulation frequency and the stimulation amplitude output by the deep brain stimulation simulation module, and the subject brain is stimulated with the deep brain stimulation current. The sensor senses the synaptic signal output from the subject brain, and extracts multiple feature values according to the synaptic signal. The reinforcement learning module outputs the stimulation frequency and the stimulation amplitude to the deep brain stimulation module through the trained deep brain stimulation neural network. In addition, the close-loop deep brain stimulation algorithm method for Parkinson's disease further includes sensing the brain cortical signal and the thalamic action potential signal of the subject brain through the sensor.

Based on the above, the close-loop deep brain stimulation algorithm system for Parkinson's disease and the close-loop deep brain stimulation algorithm system method for Parkinson's disease described in this disclosure use twin-delayed deep deterministic policy gradient (TD3) architecture as a reinforcement learning (RL) framework. Based on the Rubin-Terman neural model, the basal ganglia-thalamic (BGT) brain network is established as the training environment, including four neural nuclei: the subthalamic nucleus (STN), the external global pallidus (GPe), the internal global pallidus (GPi), and the thalamus (TH). Then, the OpenAI Gym framework is used to design the interactive interface between the environment and the reinforcement learning module, including action, state, reward mechanism, time window length, etc., so that the deep brain stimulation neural network model may find the suitable stimulation parameter (frequency and amplitude) for any input state.

In summary, the close-loop deep brain stimulation algorithm system for Parkinson's disease and the close-loop deep brain stimulation algorithm method for Parkinson's disease described in this disclosure may use machine learning (ML) to mine the hidden information in brain signals to help predict symptoms or promote electrical stimulation decisions. Moreover, the twin-delayed deep deterministic policy gradient architecture used in this disclosure, combined with the basal ganglia-thalamic virtual brain dynamic network environment, the feature extraction module, biphasic waveform, and OpenAI Gym framework design are also different from the conventional close-loop deep brain stimulation method, which achieves better thalamus relay repair effect and further saves energy consumption.

What is claimed is:

1. A close-loop deep brain stimulation algorithm system for Parkinson's disease, comprising:
a memory, storing a deep brain stimulation neural network; and
a processor, coupled to the memory, the processor comprising:
a deep brain stimulation simulation module, adapted to combine a deep brain stimulation waveform according to a stimulation frequency and a stimulation amplitude and output the deep brain stimulation waveform;
a virtual brain network module, adapted to receive the deep brain stimulation waveform to output a synaptic signal and calculate a reward parameter, and store the reward parameter to the memory;
a feature extraction module, adapted to receive the synaptic signal and extract a plurality of feature values according to the synaptic signal, and store the plurality of feature values into the memory; and
a reinforcement learning module, adapted to train the deep brain stimulation neural network based on the plurality of feature values and the reward parameter and output the stimulation frequency and the stimulation amplitude to the deep brain stimulation simulation module.

2. The close-loop deep brain stimulation algorithm system for Parkinson's disease according to claim 1, wherein the virtual brain network module is more adapted to generate a virtual brain cortical signal and a virtual thalamic action potential signal, and a thalamic error index is calculated according to the virtual brain cortical signal and the virtual thalamic action potential signal.

3. The close-loop deep brain stimulation algorithm system for Parkinson's disease according to claim 2, wherein the virtual brain network module is more adapted to calculate the reward parameter according to the thalamic error index.

4. The close-loop deep brain stimulation algorithm system for Parkinson's disease according to claim 3, wherein the reward parameter is related to a revised score, a deep brain stimulation energy expenditure penalty, a current state penalty, and a compensation score.

5. The close-loop deep brain stimulation algorithm system for Parkinson's disease according to claim 4, wherein the reward parameter is a sum of each of the revised score, the deep brain stimulation energy expenditure penalty, the current state penalty, and the compensation score multiplied by respective weight parameters.

6. The close-loop deep brain stimulation algorithm system for Parkinson's disease according to claim 1, wherein the plurality of feature values comprises Hjorth parameters, a 0 band power, and a sample entropy, wherein the Hjorth parameters comprises a Hjorth activity indicator, a Hjorth mobility indicator, and a Hjorth complexity indicator.

7. The close-loop deep brain stimulation algorithm system for Parkinson's disease according to claim 1, wherein the reinforcement learning module is a twin-delayed deep deterministic policy gradient (TD3) architecture.

8. The close-loop deep brain stimulation algorithm system for Parkinson's disease according to claim 1, wherein the deep brain stimulation waveform is a biphasic pulse wave.

9. The close-loop deep brain stimulation algorithm system for Parkinson's disease according to claim 1, further comprising:
a deep brain stimulator, connected to the deep brain stimulation simulation module and a subject brain, adapted to use the stimulation frequency and the stimulation amplitude output by the deep brain stimulation simulation module as the deep brain stimulation waveform to generate a deep brain stimulation current corresponding to the deep brain stimulation waveform, and stimulate the subject brain with the deep brain stimulation current; and
a sensor, connected to the feature extraction module and the subject brain, adapted to sense the synaptic signal output from the subject brain;
wherein the feature extraction module receives the synaptic signal output from the sensor and extracts the plurality of feature values according to the synaptic signal, the reinforcement learning module outputs the stimulation frequency and the stimulation amplitude to the deep brain stimulation module through a trained deep brain stimulation neural network.

10. The close-loop deep brain stimulation algorithm system for Parkinson's disease according to claim 9, wherein the sensor is more adapted to sense a brain cortical signal and a thalamic action potential signal of the subject brain.

11. A close-loop deep brain stimulation algorithm method for Parkinson's disease, comprising:
combining a deep brain stimulation waveform through a deep brain stimulation simulation module according to a stimulation frequency and a stimulation amplitude, and outputting the deep brain stimulation waveform;
receiving the deep brain stimulation waveform through a virtual brain network module to output a synaptic signal and calculating a reward parameter;
receiving the synaptic signal through a feature extraction module, extracting a plurality of feature values according to the synaptic signal; and
training a deep brain stimulation neural network through a reinforcement learning module based on the plurality of feature values and the reward parameter, and outputting the stimulation frequency and the stimulation amplitude to the deep brain stimulation simulation module.

12. The close-loop deep brain stimulation algorithm method for Parkinson's disease according to claim 11, further comprising:
generating a virtual brain cortical signal and a virtual thalamic action potential signal through the virtual brain network module, and calculating a thalamic error index according to the virtual brain cortical signal and the virtual thalamic action potential signal.

13. The close-loop deep brain stimulation algorithm method for Parkinson's disease according to claim 12, further comprising:
calculating the reward parameter through the virtual brain network module according to the thalamic error index.

14. The close-loop deep brain stimulation algorithm method for Parkinson's disease according to claim 13, wherein the reward parameter is related to a revised score, a deep brain stimulation energy expenditure penalty, a current state penalty, and a compensation score.

15. The close-loop deep brain stimulation algorithm method for Parkinson's disease according to claim 14, wherein the reward parameter is a sum of each of the revised score, the deep brain stimulation energy expenditure penalty, the current state penalty, and the compensation score multiplied by respective weight parameters.

16. The close-loop deep brain stimulation algorithm method for Parkinson's disease according to claim 11, wherein the plurality of feature values comprises Hjorth parameters, a β band power, and a sample entropy, wherein the Hjorth parameters comprises a Hjorth activity indicator, a Hjorth mobility indicator, and a Hjorth complexity indicator.

17. The close-loop deep brain stimulation algorithm method for Parkinson's disease according to claim 11, wherein the reinforcement learning module is a twin-delayed deep deterministic policy gradient (TD3) architecture.

18. The close-loop deep brain stimulation algorithm method for Parkinson's disease according to claim 11, wherein the deep brain stimulation waveform is a biphasic pulse wave.

19. The close-loop deep brain stimulation algorithm method for Parkinson's disease according to claim 11, further comprising:
generating a deep brain stimulation current through a deep brain stimulator according to the stimulation frequency and the stimulation amplitude output by the deep brain stimulation simulation module, and stimulating a subject brain with the deep brain stimulation current;
sensing the synaptic signal output from the subject brain through a sensor; and
receiving the synaptic signal output from the sensor through the feature extraction module and extracting the plurality of feature values according to the synaptic signal, wherein the reinforcement learning module outputs the stimulation frequency and the stimulation amplitude to the deep brain stimulation module through a trained deep brain stimulation neural network.

20. The close-loop deep brain stimulation algorithm method for Parkinson's disease according to claim 19, further comprising:
sensing a brain cortical signal and a thalamic action potential signal of the subject brain through the sensor.
